(19) United States
(12) Patent Application Publication
(10) Pub. No.: US 2011/0160624 A1
(43) Pub. Date: Jun. 30, 2011

(54) APPARATUS FOR CREATING A THERAPEUTIC SOLUTION AND DEBRIDEMENT WITH ULTRASOUND ENERGY

(75) Inventor: Eilaz Babaev, Minnetonka, MN (US)

(73) Assignee: BACOUSTICS, LLC, Minnetonka, MN (US)

(21) Appl. No.: 13/042,174

(22) Filed: Mar. 7, 2011

Related U.S. Application Data
(63) Continuation-in-part of application No. 11/777,986, filed on Jul. 13, 2007, now Pat. No. 7,901,388.

(51) Int. Cl. A61N 7/00

(52) U.S. Cl. 601/2

(57) ABSTRACT

An ultrasound surgical apparatus is provided. The apparatus is constructed from a tip mechanically coupled to an ultrasound horn containing an internal chamber. The horn is mechanical coupled to an ultrasound transducer driven by a generator. The ultrasound tip possesses at least one radial surface, a cavity, or some other form of a hollowed out area, within at least one of the radial surfaces, and a sharpened cutting member at the opening of the cavity. The horn contains an internal chamber enabling the creation of therapeutic solution that may serve as or be incorporated into a coupling medium delivered to the cavity of the tip.

### CROSS-REFERENCE TO RELATED APPLICATIONS

This application is a continuation-in-part of U.S. patent application Ser. No. 11/777,986, filed Jul. 13, 2007, the teachings of which are hereby incorporated by reference.

### BACKGROUND OF THE INVENTION

1. Field of the Invention

The present invention relates to an apparatus for debriding wounds and/or various tissues of the body such as, but not limited to, tumors, epithelial tissue, muscle, and/or cartilage with ultrasound energy while simultaneously creating a therapeutic combination with ultrasonic waves.

2. Description of the Related Art

When confronted with wounded tissue, physicians and similar practitioners of medical arts have numerous devices and methods at their disposal. Treating the wound can be simply accomplished by placing a bandage on the wound as to prevent contaminants such as, but not limited, microorganisms and dirt from entering the wound. Exposing the wound to hyperbaric oxygen may also bring about a therapeutic effect. More persistent and/or chronic wounds can be treated with repeated administrations of negative pressure therapy.

Administering pharmaceuticals to the wound may also be utilized to treat wounded tissue. A therapeutic benefit may be obtained by preventing an infection from developing in the wounded tissue. Keeping the wound in an infection free state can be accomplished by administering various anti-microbial agents such as, but not limited to, antiseptics, antibiotics, antiviral agents, antifungal agents, or any combination thereof. Administering various growth factors to the wounded tissue may also elicit a therapeutic benefit by promoting the growth of new tissue.

In extreme situations, the practitioner may have to resort to surgery to treat the wounded tissue. Grafting transplanted and/or bioengineered tissue onto the wounded may be necessary with severe wounds.

More experimental treatments, such as exposing the wounded tissue to ultraviolet light, electricity, and/or ultrasound, are also available to the practitioner. For example, U.S. Pat. Nos. 6,478,754, 6,761,729, 6,533,803, 6,569,099, 6,663,554, and 6,960,173 teach methods and devices utilizing an ultrasound generated spray to treat wounded tissues. Methods and devices utilizing indirect contact with the wounded tissue via a liquid aerosol are disclosed in U.S. Pat. Nos. 7,025,735 and 6,916,296. As taught by U.S. Patent Applications 2004/0030254 and 2006/0241470, directly contacting the wounded tissue with an ultrasonically vibrating probe may also be utilized to elicit a therapeutic effect by debriding the wound.

### BRIEF SUMMARY OF THE INVENTION

Treating severe and/or chronic wounds can be especially difficult. Successful treatment often requires the repeated removal of necrotic and/or diseased tissue by surgical debridement. The painful nature of surgical debridement, however, results in poor patient compliance. In the case of an infected and/or inflamed wound, surgical debridement procedures may be even more painful. Instead of enduring the pain of the cure, the patient chooses to live with the wound. Allowing the wound to go untreated, the patient becomes at risk for developing an infection and/or other complications. As the complications increase in severity, the patient may experience a reduced quality of life. For instance, an untreated diabetic ulcer on a patient's foot may become so painful that patient has difficulty walking.

Even when properly treated, wound healing requires that the tissue comprising the wound bed receives nutrients and other healing promoting factors. Generally, such factors are delivered to the wound bed through the circulatory system. The blood supply to wounded tissue, unfortunately, is often diminished or compromised. Consequently, the amount of healing promoting factors reaching wounded tissue is often reduced.

An ultrasound surgical apparatus capable of delivering various healing factors to a wound bed and enabling relatively pain-free wound debridement is provided. The apparatus comprises a tip mechanically coupled to an ultrasound horn. The horn is mechanically coupled to an ultrasound transducer driven by a generator. The ultrasound tip comprises at least one radial surface, a paraboloid cavity, and a sharpened cutting member at the opening of the cavity. The horn contains an internal chamber including a back wall, a front wall, and at least one side wall, at least one channel opening into the chamber, and a channel originating in the front wall of the internal chamber and terminating in the cavity of the tip. Within the internal chamber of the horn the healing factor to be delivered to the wound is mixed with a suitable carrying agent to create a therapeutic solution.

When driven or otherwise activated by the generator, the ultrasonic transducer induces ultrasonic vibrations within the tip causing ultrasonic energy to be released from the various surfaces of the tip. Directly contacting a wound and/or tissue with the vibrating tip causes the ultrasonic energy emanating from the various surfaces of the tip to enter the wound and/or tissue. The ultrasonic energy entering the wound and/or tissue reduces sensitivity to pain. Releasing ultrasonic energy into a wound and/or tissue is suspected, but not known, to change the permeability of cellular membranes to ions and/or other molecules within the extracellular environment, which may include components of the therapeutic solution. Changing membrane permeability may disrupt ionic and/or other chemical gradients relied upon by the cells to respond to painful stimuli and increase the availability of active components of the therapeutic solution.

When the transducer is activated, ultrasonic energy is also released from the walls of the paraboloid cavity. Configuring the walls of the cavity to form a parabola in at least two dimensions as to form a paraboloid may focus the ultrasonic energy emanating from the walls of the cavity towards the foci of the parabolas. If the foci of the parabolas lie outside the cavity, then the ultrasonic energy emitted from the cavity may be concentrated towards a point below the surface of the wound and/or tissue to be treated. Concentrating the ultrasonic energy emitted from the cavity at a point below the surface of the wound and/or tissue may elicited a greater change in the membrane permeability of deep cellular structures such as, but not limited to, axons and somas, further decreasing the sensation of pain in the wound and/or tissue to be treated.

As to facilitate the transmission of ultrasonic energy from the walls of the cavity to a wound and/or tissue to be treated, the therapeutic solution may fill the cavity and act as a coupling medium by conducting vibrations from the walls of the cavity to the wound and/or tissue to be treated. When the tip is ultrasonically vibrated, cavitations may form within the therapeutic solution. Additionally or in the alternative, the therapeutic solution may be atomized in the cavity into a spray. If a piezoelectric transducer is used to induce the horn and tip to vibrate approximately in resonance, then the voltage of the electrical signal driving the transducer will largely control the degree to which the therapeutic solution is cavitated and/or atomized. At low voltages, the solution within the cavity will be cavitated to a small degree. As the voltage increases, the amount of cavitations within the solution is increased. Further increasing the voltage will eventually induce atomization of the solution. Regardless of whether the solution is atomized and/or cavitated within the cavity, the presence of the solution within the cavity may couple the transmission of ultrasonic energy released from the walls of the cavity to the wound and/or tissue to be treated.

The therapeutic solution is created within the internal chamber of the ultrasound horn by the action of ultrasonic vibrations mixing the healing factor with a carrying agent. Connected to the horn's proximal end a transducer powered by a generator induces ultrasonic vibrations within the horn. Traveling down the horn from the transducer, the ultrasonic vibrations are released into the chamber from the chamber's back wall. As the vibrations travel through the chamber the healing factor and carrying agent within the chamber are agitated and thereby mixed together. Upon reaching the front wall of the chamber, the ultrasonic vibrations are reflected back into the chamber, like an echo. The ultrasonic vibrations echoing off the front wall pass through the chamber a second time, further mixing the healing factor and carrying agent.

As the vibrations travel back-and-forth within the chamber, they may strike protrusions located on the side walls of the chamber. After striking a protrusion, the vibrations are scattered about the chamber. Consequently, some the vibrations echoing off the side wall protrusions may be reflected back towards the wall of the chamber from which they originated. Some the vibrations may continue on towards the opposite the wall of the chamber. The remainder of the vibrations may travel towards another side wall of the chamber where they will be scattered once more by the protrusion. Therefore, the echoing action of ultrasonic vibrations within the chamber is enhanced by the protrusions on the side walls of the chamber. Emitting ultrasonic vibrations into the chamber from their distal facing edges, the protrusions within the inner chamber may also enhance the mixing of the healing factor and carrying agent by increasing the amount of ultrasonic vibrations within the chamber.

The protrusions may be formed in a variety of shapes such as, but not limited to, convex, spherical, triangular, rectangular, polygonal, and/or any combination thereof. The protrusions may be discrete elements. Alternatively, the protrusions may be discrete bands encircling the internal chamber. The protrusions may also spiral down the chamber similar to the threading within a nut.

The healing factor and carrying agent mixed within the chamber by the echoing and scattered ultrasonic vibrations may be any fluid and additional substance the attending physician believes will promote healing of the wound to be treated. For instance, oxygen may be utilized as the healing factor and saline as the carrying agent. Oxygen is essential for many important aspects of the healing process. For example, oxygen is required for cellular respiration, the process by which cells produce the energy needed to heal the wound. Normally oxygen delivered to tissues is transported through the blood attached to hemoglobin. As the blood travels past the cells of the body, the oxygen disassociates from hemoglobin and enters the blood plasma, a fluid similar to saline. The oxygen then diffuses from the plasma into the cells of the body. Oxygen dissociation from hemoglobin and diffusion into cells is a result of the entropic drive to achieve an equal concentration of oxygen in the blood and cells, or equilibrium. Thus, oxygen absorption by cells is driven by the difference in oxygen partial pressure between the blood and cells.

By passing oxygen and saline through the internal chamber of the horn a therapeutic solution similar to oxygen containing blood plasma can be created. When delivered into the wound bed the oxygen within the solution may diffuse into the cells of the wound bed by the entropic drive to achieve equilibrium as it would from blood plasma.

The ability of the therapeutic solution to enter the wound bed may be increased by cavitations induced within the solution and ultrasound assisted debridement with the tip. As a wound persists a diverse amount material may build up over the wound inhibiting the ability of the therapeutic solution to penetrate into the wound bed. For instance, foreign substances such as, but not limited to, dirt, debris, and/or infectious agents may collect within the wound. In the case of an infectious agent such as, but not limited to, a bacteria, a bacterial laden biofilm may develop over the wound covering healthy and/or granulation tissue. As the infection increases in severity, the wound may become covered with gangrenous tissue.

Additionally the compromised blood supply may result in ischemic tissue forming over the wound that inhibits entry of the therapeutic solution into the wound bed. Ischemia may also be the result of various conditions such as, but not limited, diabetes and/or various vascular diseases. As the ischemia persists, the tissue becomes deprived of vital nutrients required for growth and/or survival, and thus may eventually become devitalized. Failing to receive required nutrients, the devitalized tissue may eventually slip into a non-viable state. The non-viable tissue may begin a process of necrosis and/or apoptosis in which the cells of the non-viable tissue release various factors the digest and/or degrade the tissue. Destroying itself, the non-viable tissue becomes necrotic tissue. If the degradation and/or digestive process continues beyond the point of cellular death, the necrotic tissue may become slough. However, it is also possible that digestion and/or degradation stops with cellular death as to create an eschar over the wound. Regardless of how far the tissue progresses from ischemia and/or devitalization to slough and/or eschar, the dead and dying tissue may block access to the wound bed.

Material blocking entry of the therapeutic solution may also be generated by the wound itself. For instance, in response to an inflammation brought about by the presence of foreign substances and/or trauma an exudate may be secreted. As the secretion of exudate persists, the wound may become covered by various proteins and/or other molecules manufactured by the body. Secretion of a fibrinous exudate, for example, may lead to a build up fibrin over the wound. Regardless of the type of exudate secreted and/or built up over the wound, this body generated material may block access to the wound bed and should be debrided.

Various degrees of debridement may be achieved by scrapping various portions of the tip across the wound and/or tissue. For example, scrapping cutting members secured to the tip across the wound and/or tissue aggressively debrides diseased, necrotic, and/or other unwanted tissue. Cutting members may be located at the opening of the cavity and/or on the various surfaces of the tip. Ultrasonic energy released from the cutting members and/or the vibrations of the cutting members may fragment and/or emulsify the tissue being debrided, which may reduce the amount of force needed to be applied to cutting members to perform debridement. Furthermore, the ultrasonic energy released from and/or the vibrations of the cutting members permits aggressive debridement with a dull cutting edge. A moderate degree of debridement may be achieved by scrapping blunt edges within the tip across wound and/or tissue to be treated. Scrapping smooth surfaces of the tip across the wound and/or tissue to be treated may produce a mild debridement.

It should be noted and appreciated that other therapeutic benefits and/or mechanisms of actions, in addition to those listed, may be elicited by devices and methods in accordance with the present invention. The mechanisms of action presented herein are strictly theoretical and are not meant in any way to limit the scope this disclosure and/or the accompanying claims.

### BRIEF DESCRIPTION OF THE DRAWINGS

FIG. 1 is a perspective view of one embodiment of the present invention.

FIG. 2 is a perspective view of a second embodiment of the present invention.

FIG. 3 illustrates a cross-sectional view of one embodiment of an ultrasound horn and tip to be used in the present invention.

### DETAILED DESCRIPTION OF THE INVENTION

Preferred embodiments of the ultrasound apparatus comprising the present invention are illustrated throughout the figures and described in detail below.

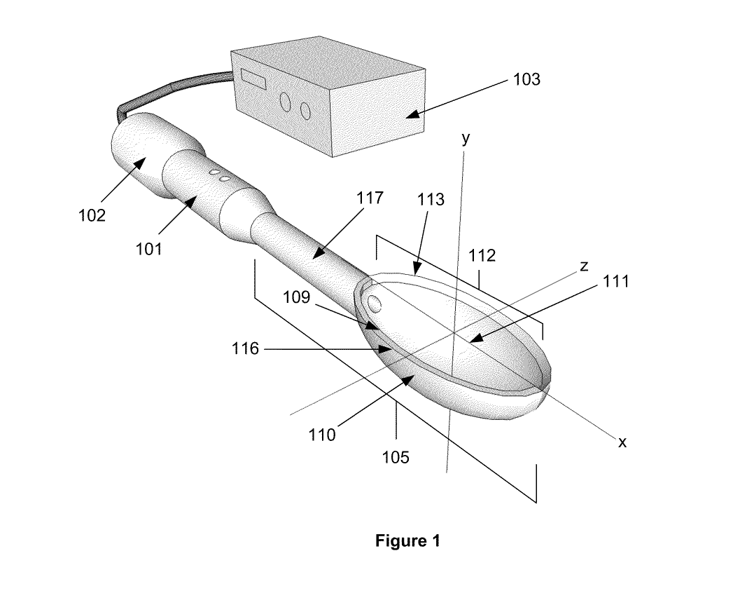

As illustrated by the embodiment depicted in FIG. 1, the apparatus comprises an ultrasound horn 101 and tip 105 attached to an ultrasound transducer 102 driven by an electrical signal produced by generator 103. As ultrasound transducers and generators are well known in the art, they need not, and will not for the sake of brevity, be described in detail herein. However, generator 103 should be capable of producing an electrical signal of a sufficient voltage to drive transducer 102 to induce horn 101 and tip 105 to vibrate approximately in resonance, with the amplitude of the vibrations being between approximately 1 micron and approximately 100 microns. Horn 101 and tip 105 may be capable of vibrating approximately in resonance at a frequency between approximately 15 kHz and approximately 3 MHz. Preferably, the combination of horn 101 and tip 105 should be capable of vibrating approximately in resonance at a frequency of approximately 30 kHz.

As illustrated by the embodiment depicted in FIG. 1, horn 101 is mechanically coupled to transducer 102 and tip 105. Mechanically coupled to transducer 102 and tip 105, horn 101 transmits ultrasonic vibrations generated by transducer 102 to tip 105. Horn 101 may be mechanically coupled to transducer 102 by mechanically attaching (for example securing with a threaded connection), adhesively attaching, and/or welding it to transducer 102. Alternatively, horn 101 and transducer 102 may be a single piece. Likewise, horn 101 may be mechanically coupled to tip 105 by mechanically attaching (for example securing with a threaded connection), adhesively attaching, and/or welding it to tip 105. Alternatively, horn 101 and tip 105 may be a single piece. As to facilitate the mechanical attachment of horn 101 to tip 105, tip 105 may, but need not, posses a shaft 117 or equivalent extension providing a point of attachment.

As illustrated by the embodiment depicted in FIG. 1, tip 105 comprises radial surfaces 109 and 110, a paraboloid cavity 111 containing an opening 112 within the radial surface 109 and not oriented orthogonal to the longitudinal axis of horn 101, and a sharpened cutting member 113 at opening 112. Though not illustrated, it would be possible for radial surface 109 to extend to opening 112 such that cavity 111 would open in radial surface 109. Providing a smooth edge that may be scrapped across a wound and/or tissue, radial surface 110 enables mild debridement. A moderate degree of debridement may be achieved by scrapping blunt edge 116 formed by the intersection of radial surfaces 109 and 110 across the wound and/or tissue to be treated. Scrapping cutting member 113 over a wound and/or tissue permits aggressive debridement. Though the particular cutting member 113 illustrated in FIG. 1 is formed by sharpening a region of radial surface 109 into an edge, the cutting member at the opening of the cavity need not be so fashioned. The cutting member may be a separate piece secured to a surface of tip 105 into which cavity 111 opens.

Aggressive debridement may also be achieved by scrapping a cutting member attached a surface of the tip away from opening 112 across the wound and/or tissue to be treated. Though sharpened to some degree, cutting members need not be so sharp as to cut and/or debride in the absence of ultrasonic vibrations. A plurality of cutting members may be attached to a surface of tip as to give the surface a rough and/or jagged appearance. Furthermore, cutting members attached to the surfaces of tip comprising a sharpened edge may run in any direction, and need not run the length of the surface to which they are attached.

Tip 105, as illustrated in FIG. 1, may contain a region that is wider than the shaft 117 and/or horn 101 in two dimensions (y and z) oriented orthogonal to the longitudinal axis of shaft 117 and/or horn 101. Though not illustrated, it should be readily appreciated by those of ordinary skill in the art that tip 105 may be constructed as to possess a region wider than shaft 104 in only one dimension oriented orthogonal to longitudinal axis shaft 117 and/or horn 101. Furthermore, tip 105 may also be constructed as to lack any region wider than shaft 117 and/or horn 101 in a dimension oriented orthogonal to the longitudinal axis of either structure.

Radial surfaces 109 and 110 of the illustrated embodiment of tip 105 form a parabola in at least two dimensions. As such, the illustrated embodiment of tip 105 is characterized by a general paraboloid structure. The plane of opening 112 need not be parallel to the longitudinal axis 115 of horn 101.

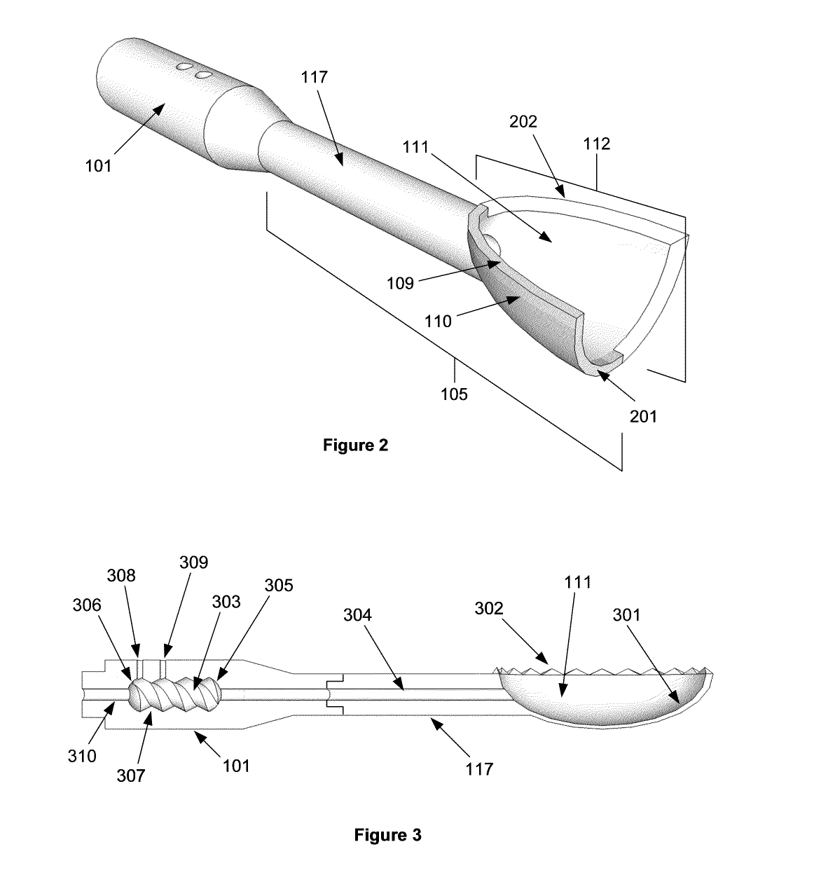

It should be appreciated from the embodiment depicted FIG. 2 that tip 105 may be comprised of more than radial surfaces. For example, as illustrated, tip 105 may also contain a distal surface 201. In the depicted embodiment, opening 112 of cavity 111 extends from distal surface 201 to radial surface 109. The depicted cutting member 202 at the opening of cavity 111 is formed by sharpening a region of distal surface 201. As with the embodiment depicted in FIG. 1, cutting member 202 may be a separate piece secured to radial surface 109, distal surface 201, and/or any other surface into which cavity 111 opens. Furthermore, the cutting member however fashioned and/or formed need not extend onto an additional surface from that on which it originates.

FIG. 3 illustrates a cross-sectional view of one embodiment of an ultrasound horn and tip to be used in the present invention. As illustrated, wall 301 of cavity 111 may form a multidimensional parabola such that cavity 111 is characterized by a general paraboloid configuration. In the embodiment illustrated in FIG. 3, the plurality of sharpened cutting members 302 encircling opening 112 of cavity 111 enables aggressive debridement. Though sharpened to some degree, cutting members 302 need not be so sharp as to cut and/or debride in the absence of ultrasonic vibrations.

As to facilitate the transmission of ultrasonic energy emanating from the wall 301 of cavity 111 when a wound and/or tissue is debrided with a cutting member, edge, and/or surface at the opening of cavity 111, cavity 111 may be filled with a coupling medium. Filling cavity 111 with a coupling medium may be accomplished by delivering a therapeutic solution created in internal chamber 303 of horn 101 through channel 304 originating in front wall 305 of chamber 303 and opening within cavity 111.

When horn 101 is induced to vibrate back wall 306 of chamber 303 oscillates back-and-forth inducing the release of ultrasonic vibrations into the fluids inside chamber 303. Positioning back wall 306 such that at least one point on back wall 104 lies approximately on an antinode (point of maximum deflection) of the ultrasonic vibrations of horn 101 may maximize the amount and/or amplitude of the ultrasonic vibrations emitted into the fluids in chamber 303. Preferably, the center of back wall 306 lies approximately on an antinode of the ultrasonic vibrations. The ultrasonic vibrations emanating from back wall 306 travel towards the front wall 305 of chamber 303. When the ultrasonic vibrations strike front wall 305 they echo off it, and thus are reflected back into chamber 303. The reflected ultrasonic vibrations then travel towards back wall 306. Traveling towards front wall 305 and then echoing back towards back wall 306, the ultrasonic vibrations travel back and forth through chamber 303 in an echoing pattern. As to maximize the echoing of vibrations off front wall 305, it may be desirable to position front wall 305 such that at least one point on it lies on an antinode of the ultrasonic vibrations. Preferably, the center of front wall 305 lies approximately on an antinode of the ultrasonic vibrations.

The incorporation of protrusions 307 enhances ultrasonic echoing within chamber 303 by increasing the amount of ultrasonic vibrations emitted into chamber 303 and/or by providing a larger surface area from which ultrasonic vibrations echo. The distal or front facing edges of protrusions 307 may emit ultrasonic vibrations into chamber 303 when the ultrasound transducer 102 is activated. The proximal, or rear facing, and front facing edges of protrusions 307 reflect ultrasonic waves striking the protrusions. Emitting and/or reflecting ultrasonic vibrations into chamber 303, protrusions 307 increase the complexity of the echoing pattern of the ultrasonic vibrations within chamber 303. The specific protrusions 307 depicted in FIG. 3 comprise a triangular shape and spiral down the chamber similar to the threading within a nut. The protrusions may be formed in a variety of shapes such as, but not limited to, convex, spherical, triangular, rectangular, polygonal, and/or any combination thereof. In the alternative or in combination to spiraling down the chamber, the protrusions may be discrete bands encircling the chamber. In combination or in the alternative, the protrusions may be discrete elements secured to a side wall of chamber that do not encircle the chamber. In the alternative or in combination, the protrusions may be integral with side wall or walls of the chamber.

The healing factor and carrying agent to be mixed to create the therapeutic solution enter chamber 303 of the embodiment depicted through at least one channel opening into chamber 303. As illustrated by channels 308 and 309, the healing factor and carrying agent may enter chamber 303 via channels opening in a side wall of chamber 303. In the alternative or in combination, the healing factor or carrying agent may enter chamber 303 through a channel 310 opening in back wall 306. Preferably, channels 308 and 309 encompass a node of the ultrasonic vibrations traveling down the length of the horn 101 and/or emanating from back wall 306. Upon entering chamber 303, the materials are mixed to create the therapeutic solution as it flows through chamber 303. The therapeutic solution created then exits chamber 303 through channel 304 and enters cavity 111

Alternative embodiments of an ultrasound horn 101 in accordance with the present invention may possess a single channel 109 opening within side wall of chamber 103. If multiple channels opening within a side wall are utilized, they may be aligned along the central axis of horn 101 on different platans, as depicted in FIG. 3. Alternatively, the channels may be located on the same platan.

A single channel may be used to deliver the carrying agent and healing factor to be mixed to create the therapeutic solution into chamber 303.

In the embodiment depicted in FIG. 3 back wall 305 and front wall 306 have been fashioned to form ultrasonic lenses with concave portions. If the concave portions of lens within back wall 306 form an overall parabolic configuration in at least two dimensions, then the ultrasonic vibrations depicted by arrows emanating from the lens travel in a pattern of convergence towards the parabola's focus. As the ultrasonic vibrations converge at the focus the ultrasonic energy carried by the vibrations may become focused or concentrated. After converging at the focus, the ultrasonic vibrations diverge and continue towards front wall 305. After striking the concave portions of the lens within front wall 305 the ultrasonic vibrations are reflected back into chamber 303. If concave portions of the lens within front wall 305 form an overall parabolic configuration in at least two dimensions the ultrasonic vibrations echoing back into chamber 303 may travel in a pattern of convergence towards the parabola's focus. Converging as they travel towards front wall 305 and then again as they echo back towards back wall 306, the ultrasonic vibrations travel back and forth through chamber 303 in a converging echoing pattern.

In addition to focusing the ultrasonic vibrations and/or the ultrasonic energy they carry, ultrasonic lenses within back wall 306 and front wall 305 direct the ultrasonic vibrations towards the side walls of chamber 303. As such, an increased amount of ultrasonic vibrations emanating from back wall 306 and/or reflecting off front wall 305 strike the side wall and become scattered by protrusions 127.

The parabolas formed by the concave portions of lenses within back wall 306 and front wall 305 may have a common focus. In the alternative, the parabolas may have different foci. However, by sharing a common focus, the ultrasonic vibrations emanating and/or echoing off the parabolas and/or the energy the vibrations carry may become focused at the shared focus. The materials passing through chamber 303 are therefore exposed to the greatest concentration of the ultrasonic agitation at the shared focus. Consequently, the ultrasonically induced mixing of the fluids is greatest at the shared focus. Positioning the shared focus, or any other focus of a parabola formed by the concave portions within back wall 306 and front wall 305, at point downstream of the entry of at least two materials into chamber 303 may maximize the mixing of the materials.

It is possible that lenses formed within back wall 306 or front wall 305 contain convex portions. Ultrasonic vibrations emanating from such convex portions travel in a dispersed reflecting pattern towards the opposite wall in the following manner: The ultrasonic vibrations are first directed towards the side walls at varying angles of trajectory. The ultrasonic vibrations then reflect off a side wall and become scattered by protrusions 127. The scattered ultrasonic vibrations may then travel back towards wall from which they originated, continue on towards the opposite wall, and/or become scattered again by protrusions 127 on another side wall region.

It should be appreciated that the configuration of the chamber's front wall lens need not match the configuration of the chamber's back wall lens. Furthermore, the lenses within the front and/or back walls of the chamber may comprise any combination of the above mentioned configurations such as, but not limited to, an outer concave portion encircling an inner convex portion.

After being delivered to cavity 111 the created therapeutic solution to be used as or incorporated into the coupling medium cavitations may be formed in coupling medium and/or therapeutic solution if the generator driving transducer 102 produces an electrical signal of a sufficient voltage. If the voltage of the electrical signal is further increased, the coupling medium and/or therapeutic solution will atomize into spray. If the walls of cavity 111 form a parabola in at least two dimensions, the atomized spray and/or the ultrasonic energy emanating from the walls of cavity 111 may be focused towards the foci of the parabolas, which may be located inside, outside of cavity 111 or in the plane of opening 112. Positioning a focus outside cavity 111 may enable the ultrasonic energy emitted from the walls of cavity 111 to be concentrated towards a point below the surface of the wound and/or tissue being treated.

It should be appreciated that elements described with singular articles such as “a”, “an”, and/or “the” and/or otherwise described singularly may be used in plurality. It should also be appreciated that elements described in plurality may be used singularly.

Although specific embodiments of apparatuses and methods have been illustrated and described herein, it will be appreciated by those of ordinary skill in the art that any arrangement, combination, and/or sequence that is calculated to achieve the same purpose may be substituted for the specific embodiments shown. It is to be understood that the above description is intended to be illustrative and not restrictive. Combinations of the above embodiments and other embodiments as wells as combinations and sequences of the above methods and other methods of use will be apparent to individuals possessing skill in the art upon review of the present disclosure.

The scope of the claimed apparatus and methods should be determined with reference to the appended claims, along with the full scope of equivalents to which such claims are entitled.

1. An apparatus comprising a:
a. an ultrasound horn containing:
i. an internal chamber having a back wall, a front wall and at least one side wall extending between the back wall and the front wall;
ii. at least one channel opening into the internal chamber; and
iii. at least one protrusion on a side wall of the chamber extending into the chamber;
b. a tip attached to the distal end of the ultrasound horn containing:
i. at least one radial surface;
ii. a paraboloid cavity containing at least one wall and an opening within at least one of the radial surfaces of the tip wherein the plane of the opening is not orientated orthogonal to the longitudinal axis of the horn; and
iii. a sharpened cutting member or plurality of sharpened cutting members at the opening of the cavity; and
c. a channel originating in the front wall of the internal chamber and opening within a wall of the cavity of the tip.

2. The apparatus of claim 1 further comprising a coupling medium delivered to the cavity of the tip through the channel riginating in the front wall of the internal chamber.

3. The apparatus of claim 1 further comprising a region of the tip wider than the horn in at least one dimension oriented orthogonal to the longitudinal axis of the horn.

4. The apparatus of claim 1 further comprising a shaft connecting the horn and tip.

5. The apparatus of claim 4 further comprising a region of the tip wider than the shaft in at least one dimension oriented orthogonal to the longitudinal axis of the shaft.

6. The apparatus according to claim 1 characterized by being capable of vibrating approximately in resonance at a frequency between approximately 15 kHz and approximately 3 MHz.

7. The apparatus of claim 1 characterized by being capable of vibrating approximately in resonance at a frequency of approximately 30 kHz.

8. The apparatus of claim 1 further comprising a parabola formed by the radial surfaces of the tip in at least two dimensions.

9. The apparatus of claim 1 characterized by a focus of the paraboloid cavity of the tip lying outside the cavity.

10. The apparatus of claim 1 further comprising at least one distal surface on the tip.

11. The apparatus of claim 10 further comprising a distal opening to the cavity of the tip within at least one of the distal surfaces on the tip.

12. The apparatus of claim 11 further comprising an additional cutting member on the distal surface on the tip at the distal opening of the cavity.

13. The apparatus of claim 1 further comprising an ultrasound transducer attached to the proximal end of the horn.

14. The apparatus according to claim 13 characterized by the generator being capable of producing an electrical signal of a voltage sufficient to induce the combination of the horn and tip to vibrate approximately in resonance with the amplitude of the vibrations being between approximately 1 micron and approximately 100 microns.

15. The apparatus according to claim 13 characterized by the generator being capable of producing an electrical signal of a voltage sufficient to induce the substructure formed by the combination of the horn and tip to vibrate approximately in resonance with the amplitude of the vibrations being approximately 100 microns.

16. The apparatus according to claim 13 characterized by the generator being capable of producing an electrical signal of a voltage sufficient to induce cavitations within a coupling medium within the cavity of the tip.

17. The apparatus according to claim 13 characterized by the generator being capable of producing an electrical signal of a voltage sufficient to atomize a coupling medium within the cavity of the tip.

18. The apparatus according to claim 1 further comprising an ultrasonic lens within the back wall of the chamber.

19. The apparatus according to claim 18 further comprising one or a plurality of concave portions within the lens within the back wall that form an overall parabolic configuration.

20. The apparatus according to claim 18 further comprising at least one convex portion within the lens within the back wall.

21. The apparatus according to claim 1 further comprising an ultrasonic lens within the front wall of the chamber.

22. The apparatus according to claim 21 further comprising one or a plurality of concave portions within the lens within the front wall that form an overall parabolic configuration.

23. The apparatus according to claim 21 further comprising at least one convex portion within the lens within the front wall.
